(19) United States
(12) Patent Application Publication
(10) Pub. No.: US 2016/0289617 A1
(43) Pub. Date: Oct. 6, 2016

(54) GAS-INFUSED FLUIDS AND METHODS OF MAKING AND USING SAME

(71) Applicant: Prosper Brands LLC, Brooklyn, NY (US)

(72) Inventors: Max MacKenzie, Brooklyn, NY (US); John H. Archibald, Oakville (CA)

(21) Appl. No.: 14/933,615

(22) Filed: Nov. 5, 2015

Related U.S. Application Data
(60) Provisional application No. 62/075,706, filed on Nov. 5, 2014.

(51) Int. Cl. C12G 3/04; B01F 15/06; C12G 1/06; B01F 3/04; C12C 11/11

(52) U.S. Cl. CPC C12G 3/04; B01F 3/04787; C12C 11/11; C12G 1/06; B01F 15/06; B01F 3/04269; A61M 5/1407

(57) ABSTRACT

The present disclosure provides fluids, such as a distilled spirit, comprising at least about 25 ppm of an infused gas. Systems and methods for producing such gas-infused fluids are also provided.

### PRIORITY CLAIM

This application claims priority to U.S. Provisional Patent Application No. 62/075,706, filed Nov. 5, 2014, the entire contents of which is incorporated herein by reference and relied upon.

### TECHNICAL FIELD

This invention relates to the field of gas-infused fluids including, for example, oxygen-infused beverages, oxygen-infused therapeutic fluids, nitrogen-infused beverages, nitrogen-infused therapeutic fluids, carbon dioxide infused beverages, carbon dioxide infused therapeutic fluids, and other gas infused fluids.

### BACKGROUND

The alcoholic beverage industry including distilled spirits, wines and beers, has traditionally used aging and other methods to influence flavor profiles and reduce harsh fermentation by-products in finished products. These processes are time intensive, often unpredictable, and require storage of large quantities of the beverage.

There have previously been attempts to saturate alcoholic beverages with oxygen to accelerate aging and improve flavor profiles. To date, no oxygen saturation has been able to maintain a saturated beverage above 30 ppm of oxygen. As such the results have been less than desirable.

### SUMMARY

The present invention is directed to a system and method for infusing gases such as oxygen, nitrogen, or carbon dioxide into a beverage, including into an alcoholic beverage such as a distilled spirit, wine, hard cider or beer.

In one example implementation of the present invention, a system for producing oxygen infused distilled spirits comprises a compressed oxygen cylinder, a gas infusion chamber in communication with the compressed oxygen cylinder, wherein the infusion chamber comprises a micromembrane having a pore channel diameter of between 0.05 and 5.0 μm, and a distilled spirit within the gas infusion chamber wherein the distilled spirit comprises an oxygen saturation greater than 30 ppm.

In yet another example implementation of the present invention a method of infusing oxygen in a distilled spirit comprises the steps of: (a) containing a distilled spirit within an oxygen infusion chamber; wherein the oxygen infusion chamber comprises a micromembrane having a pore channel diameter of between 0.05 and 5.0 μm; (b) pressurizing the oxygen infusion chamber with pure oxygen to an internal pressure of between 15 psi and 100 psi; (c) saturating the distilled spirit with oxygen until a level greater than 30 ppm oxygen is reached; and (d) removing the oxygen saturated distilled spirit from the oxygen infusion chamber.

In still another example implementation of the present invention, a system for producing a gas infused fluid comprises a compressed oxygen source, a gas infusion chamber for receiving the fluid, wherein the gas infusion chamber is in communication with the compressed oxygen cylinder; and a micromembrane in the gas infusion chamber, wherein the micromembrane has a pore channel diameter of about 0.05 μm to about 5.0 μm. The fluid is a beverage. The beverage comprises ethanol. The fluid is a distilled spirit. The distilled spirit is selected from the group consisting of: gin, rum, bourbon, cognac, tequila, whiskey, brandy, grappa, vodka, and a liqueur. The beverage is beer. The beverage is wine. The beverage is a nutritional beverage. The fluid is a therapeutic fluid. The fluid comprises at least about 15 ppm of oxygen, at least about 25 ppm of oxygen, at least about 30 ppm of oxygen, at least about 50 ppm of oxygen, at least about 75 ppm of oxygen. at least about 100 ppm of oxygen, at least about 150 ppm of oxygen, at least about 200 ppm of oxygen. The fluid comprises more than 200 ppm of oxygen. The system comprises a compressed nitrogen source. The system comprises a fluid supply pump in communication with the gas infusion chamber. The system comprises a holding chamber in communication with the gas infusion chamber. The gas infusion chamber is housed within the holding chamber. The system comprises a supply pump in communication with the holding chamber and the gas infusion chamber. The system comprises a distiller in communication with the gas infusion chamber. The system comprises a chiller in thermal communication with a supply line, wherein the supply line is in fluid communication with the gas infusion chamber.

In a further example implementation of the present invention, a method of infusing oxygen in a distilled spirit comprises the step of: (a) containing a fluid within a gas infusion chamber; wherein the gas infusion chamber comprises a micromembrane having a pore channel diameter of about 0.05 μm to about 5.0 μm; (b) pressurizing the gas infusion chamber with oxygen gas; (c) contacting the fluid with the oxygen gas in the gas infusion chamber to provide an oxygenated fluid comprising at least about 25 ppm of oxygen; and (d) removing the oxygenated fluid from the gas infusion chamber. The gas infusion chamber is pressurized to about 15 psi to about 100 psi. The fluid in the gas infusion chamber is circulated. The step of circulating comprises passing the fluid from a holding tank through the gas infusion chamber a plurality of times. The method further comprising releasing the oxygen gas from the gas infusion chamber and pressurizing the gas infusion chamber with a second gas. The second gas is nitrogen or carbon dioxide. The further comprising releasing the second gas from the gas infusion chamber and pressurizing the gas infusion chamber with a third gas, wherein the third gas is different from the second gas. The third gas is carbon dioxide or nitrogen. The method further comprises heating the fluid. The method further comprises cooling the fluid.

Various embodiments and implementations of the present invention have one or more of the following advantages: accelerated aging of whiskey and other dark spirits; smoother tasting spirits and improved finish and flavor profile for a younger spirit; reduced cost of goods: less storage space needed for aging of spirits, reduced number of barrels needed for long aging process, and less money tied up in inventory for years; increased speed to market of new brands of spirits; improved ability to forecast supply chain needs; ability to manage flavor profiles; ability to replicate barrel aging flavor profiles; and the ability to infuse designer flavors.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a system diagram of an example implementation of the present invention.

FIG. 2 is a system diagram of another example implementation of the present invention.

FIG. 3 is a system diagram of another example implementation of the present invention.

FIG. 4 shows levels of acetaldehyde, methanol and ethyl acetate levels in white whiskey when untreated (“Control”) or infused with oxygen in the presence of oak chips for 24, 60, or 120 hours according to embodiments of the present disclosure.

FIG. 5 shows levels of various fusel oils in white whiskey when untreated (“Control”) or infused with oxygen in the presence of oak chips for 24, 60, or 120 hours according to embodiments of the present disclosure.

FIG. 6 shows levels of various aroma compounds in white whiskey when untreated (“Control”) or infused with oxygen in the presence of oak chips for 24, 60, or 120 hours according to embodiments of the present disclosure.

FIG. 7 shows the level of furfural in white whiskey when untreated (“Control”) or infused with oxygen in the presence of oak chips for 24, 60, or 120 hours according to embodiments of the present disclosure.

FIG. 8 shows levels of acetaldehyde, ethyl acetate, ethanol (“alcohol”) and methanol in commercially available vodka, gin and rum distilled beverages before (“CT”) and after (“T”) a 24-hour or 36-hour oxygen infusion process consistent with embodiments of the present disclosure.

FIG. 9 shows levels of acetaldehyde, ethyl acetate, ethanol (“alcohol”) and methanol in commercially available tequila distilled beverages before (“CT”) and after (“T”) a 24-hour or 36-hour oxygen infusion process consistent with embodiments of the present disclosure.

FIG. 10 shows levels of various fusel oils in commercially available vodka, gin and rum distilled beverages before (“CT”) and after (“T”) a 24-hour or 36-hour oxygen infusion process consistent with embodiments of the present disclosure.

FIG. 11 shows levels of various fusel oils in commercially available tequila distilled beverages before (“CT”) and after (“T”) a 24-hour or 36-hour oxygen infusion process consistent with embodiments of the present disclosure.

Like reference symbols in the various drawings indicate like elements.

### DETAILED DESCRIPTION

Various implementations of the present invention are directed to a system and method for infusing gases, such as oxygen, into products for consumption by or administration to a human, such as a beverage. Some aspects of the present invention relate to infusion of oxygen or nitrogen into alcoholic beverages. Oxygen infused distilled spirits, wines and beers using the systems and methods of the present invention have shown to have an improved palette, flavor profile, smoothness, and overall flavor presentation to beverages lacking the oxygen infusion treatment. Indeed, oxygen infusion of such alcoholic beverages can approximate the flavor profiles of alcoholic beverages that have been aged in barrels or casks without having to endure the lengthy aging process, which can often last between two and twenty-one years or more, depending on the spirit, wine, or beverage. In addition to the improved flavor profile, oxygen infusion of distilled spirits can lessen the adverse impacts of body detoxification after intoxication (the hangover effect).

In other embodiments, the present invention is directed to a gas-infused fluid for administration to a human. In some such embodiments, the gas-infused fluid is for infusion into a human subject, such as a volume expander, blood or a component thereof (e.g., plasma), a saline solution, a buffer solution, a dialysis fluid, or a nutritional fluid (e.g., a geriatric drink, an infant drink, or a parental nutritional fluid).

Various implementations of the present invention include a production process for gas infusing spirits, wine, beer, and beverage ingredients. The process can infuse beverages and ingredients with high levels of stable dissolved oxygen or nitrogen up to 225 parts per million (ppm). The positive benefits of highly oxygenated alcoholic beverages include: accelerated aging of whiskey and other spirits, removal of objectionable fermentation by-products, improved flavor profile, and for wines, increased shelf life, and improved flavor quality.

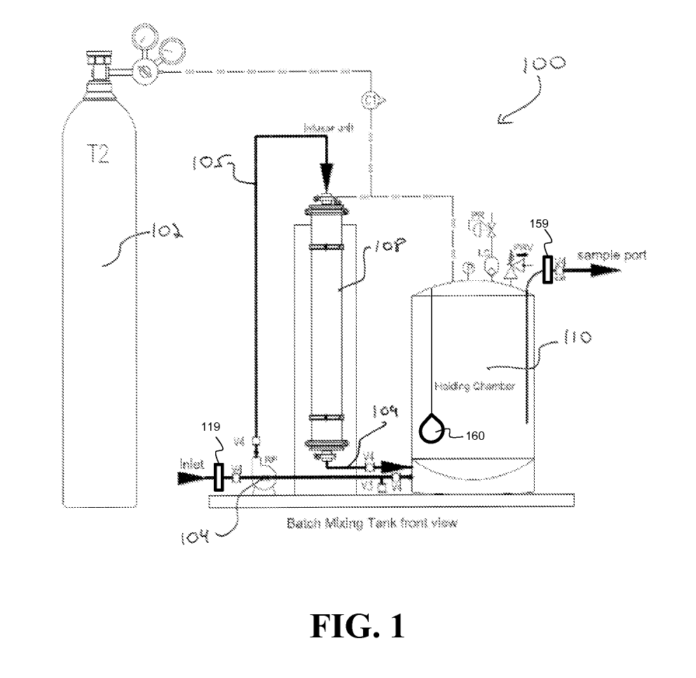

FIG. 1 illustrates an example system for oxygen or nitrogen infusion of distilled spirits, wines or beer. System 100 comprises compressed gas source 102, fluid supply pump 104, fluid supply line 105, gas infusion, infused gas discharge line 109, and holding chamber 110. FIG. 1 may optionally also include one or more filters 119,159, and/or a wood chip teabag 160.

In an example implementation, a fluid such as an alcoholic beverage (e.g., a clear or dark distilled spirit, wine, hard cider, or beer) is transferred to the gas infusion chamber 108 either by directly pouring the alcoholic beverage into the gas infusion chamber 108 or via supply pump 104 and supply line 105. Optionally, the fluid may pass thru a filter 119 (e.g., a carbon filter) before entering the gas infusion chamber 108. Pressurized gas, such as oxygen or nitrogen, is transferred from the compressed gas source 102 to the gas infusion chamber 108 to achieve a total pressure within the chamber greater than one atmosphere. Total pressure within the gas infusion chamber 108 may be at least about 15 psi, for example about 15 psi to about 120 psi, about 25 psi to about 100 psi, about 20 psi, about 30 psi, about 40 psi, about 50 psi, about 60 psi, about 70 psi, about 80 psi, about 90 psi, about 100 psi, about 110 psi, about or about 120 psi). In some embodiments, the total pressure within the gas infusion chamber 108 is about 15 psi to about 120 psi. The increased gas pressure causes an infusion of the gas into the fluid. Optionally, circulating the fluid within the gas infusion chamber 108 may provide more efficient infusion of the gas into the fluid, and may in some embodiments be accomplished by recirculating the fluid through the gas infusion chamber 108 via supply pump 104 and supply line 105. The resulting gas-infused fluid may have a gas level of at least about 25 ppm to about 250 ppm or more (e.g., about 40 ppm, 50 ppm, 60 ppm, 70 ppm, 80 ppm, 90 ppm 100 ppm, 110 ppm, 120 ppm, 130 ppm, 140 ppm, 150 ppm, 160 ppm, 170 ppm, 180 ppm, 190 ppm, 200 ppm, 210 ppm, 220 ppm, 230 ppm, 240 ppm, or 250 ppm). In one embodiment, the fluid has a gas level of about 160 ppm.

Once the alcoholic beverage is infused with the gas, it may be transferred to the holding chamber 110. Optionally, the holding chamber 110 may include a wood chip teabag 160, which may include wood chips selected to impart various flavor compounds to the gas-infused fluid. The holding chamber 110 may be maintained at a pressure greater than one atmosphere in an environment consistent with the saturated gas in the beverage to prevent seeping of the gas from the solution. However, it has been found that infusion of gases into alcoholic beverages in accordance with the present invention results in a stable supersaturated solution without a lasting effervescence quality. As such, the holding chamber 110 does not need to be maintained under pressure in order to maintain the infused gas level of at least 25 ppm.

Gas infusion chamber 110 comprises a microporous hydrophobic hollow fibre membrane having a pore pathway diameter of about 0.01 μm to about 5 μm (hereinafter a “micromembrane”). Various embodiments of the gas infusion chamber are described in U.S. Pat. Nos. 6,209,855 and 7,537,200, the entire contents of each of which are incorporated herein by reference.

The oxygen-infused fluid may be removed from the holding chamber 110 through a port, which may optionally include a filter 159 such as a carbon filter.

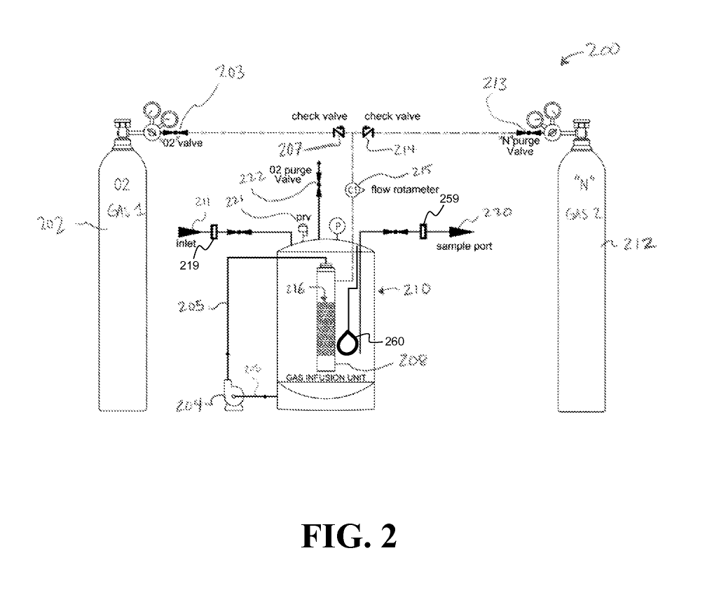

Referring now to FIG. 2, another example system 200 of the present invention comprises a compressed gas source 202, a gas infusion chamber 208, a holding tank 210, and a second compressed gas source 212. A fluid is introduced to the holding tank 210 through inlet 211, and optionally after passing through a filter 219 (e.g., a carbon filter). The gas infusion chamber includes a micromembrane 216 as described above with respect to FIG. 1.

In this embodiment, the gas infusion chamber 208 is housed inside the holding tank 210. In some embodiments, the holding tank 210 may include a wood chip teabag 260. Under gas pressure provided by the compressed gas source 202 (e.g., a compressed oxygen cylinder), fluid from the holding tank 210 is fed through the gas infusion chamber 208 via pathway 205 and incorporated pump 204. In some embodiments, a mixture of gas from the compressed gas source 202 and a second gas from second gas source 212 may be provided to the holding tank 210. In such embodiments, both check valves 207,214 may be opened to allow both gases to enter the holding tank 210. The relative amounts of the two gases may be controlled using valves 203 and 213. Flow of the gas(es) may be monitored or measured using a flow rotameter 215. Holding tank 210 may further include a pressure release valve 221 or other safety valve.

Alternatively, gas infusion of the fluid may occur first under only one gas, for example oxygen from the compressed gas source 202. In such embodiments, the second check valve 214 may be closed while first check valve 207 may be opened to allow only the first gas to enter the holding tank 210. After infusing the fluid with the first gas, excess gas pressure may be relieved by opening purge valve 220. Thereafter, a second gas may be introduced to the holding tank 210 by closing first check valve 207 and opening second check valve 214. Optionally, the purge valve 222 may remain open for a sufficient time to enable replacement of all or substantially all of the first gas in the holding tank 210 with the second gas from the second compressed gas source 212. Thereafter, the purge valve 222 may be closed if desired to enable the pressure of the second gas to increase in the holding tank 210. Optionally, the fluid may be recirculated through the gas infusion chamber 208 in the presence of the second gas, if desired.

In some embodiments, the first gas is oxygen and the second gas is nitrogen. In such embodiments, the introduction of nitrogen gas after infusing a fluid with oxygen may serve to quench oxidation reactions (e.g., overoxidation) that occur under increased O₂ pressure in the holding tank 210 and/or in the gas infusion chamber 208. In some such embodiments, the fluid may optionally be beer, wine, or a distilled spirit.

In other embodiments, the first gas is oxygen and the second gas includes carbon dioxide. In such embodiments, the infusion of carbon dioxide after infusing with oxygen may increase an effervescent property of the fluid. In some such embodiments, the fluid may optionally be beer or wine (e.g., a sparkling wine).

In some embodiments, the fluid may be treated with a third gas from a third compressed gas source. In some embodiments, the third gas is different from the second gas. For example, a fluid may initially be infused with oxygen and thereafter treated with a second gas that is nitrogen or carbon dioxide, followed by treatment with a third gas that is carbon dioxide (e.g., if the second gas is nitrogen) or nitrogen (e.g., if the second gas is carbon dioxide).

After gas infusion of the fluid is complete, the gas-infused fluid may be removed from the holding tank 210 via port 220, optionally after passing through a filter 259, such as a carbon filter.

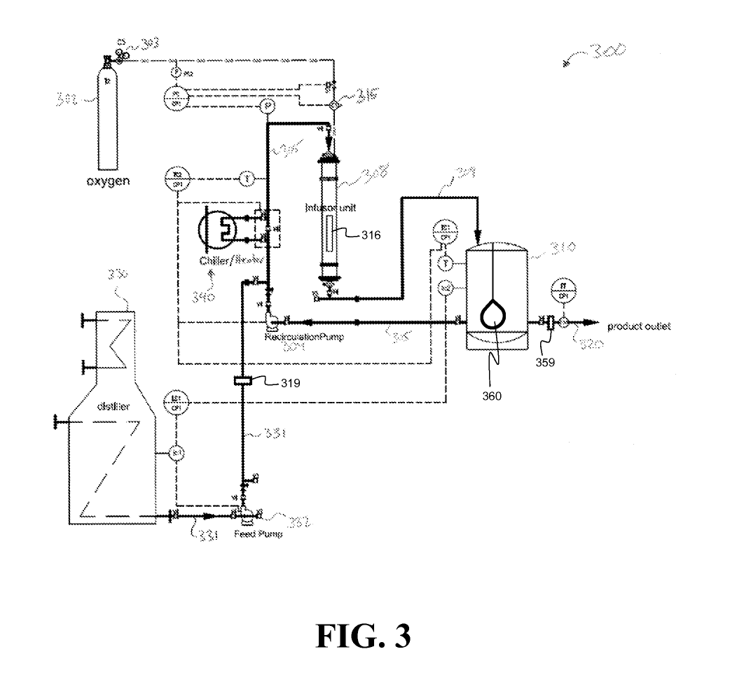

Referring now to FIG. 3, a system 300 according to another embodiment of the present disclosure comprises a compressed gas source 302, a gas infusion chamber 308, a fermentation tank 310, a distiller 330, and a chiller and/or heater 340.

Similar to systems 100 and 200, system 300 is capable of infusing a fluid with a gas. In this embodiment, however, the fluid is a distilled spirit produced by distiller 330. The distilled spirit is removed from the distiller 330 and introduced into the gas infusion chamber 308 via pathway 331 and optional incorporated pump 332. Optionally, pathway 331 may include a filter 319, such as a carbon filter. Once in the infusion unit, gas is infused into the fluid in substantially the same manner as described above with respect to FIGS. 1-2. More specifically, gas from the compressed gas source 302 is provided to the gas infusion chamber 308 and incorporated micromembrane (not shown) through valve/regulator 303. The flow of gas may be monitored (e.g., measured) using flow meter 315. After flowing through the gas infusion chamber 308, the fluid is fed into fermenter 310 via pathway 309. The fermenter 310 may optionally include a wood chip teabag 360 for imparting flavor compounds into the fluid. From the fermenter 310, the fluid may be recirculated from the fermenter 310 to the gas infusion chamber 308 via pathway 305 and incorporated pump 304. The fluid may be chilled or heated, as desired, by the chiller/heater 340, which is in thermal communication with the fluid. The chiller/heater 340 may be positioned at any suitable location along the fluid pathway (e.g., at any suitable location of pathways 331, 305 and/or 309). In the embodiment shown in FIG. 3, for example, the chiller/heater 340 is located along pathway 305 downstream of pathway 331. In this configuration, the chiller/heater 340 may be used to alter the temperature of fluid fed from the distiller 330 and/or fluid fed from the fermenter 310 before the fluid enters (or reenters) the gas infusion chamber 308. Once the fluid has obtained a desired gas infusion level (e.g., at least about 25 ppm of oxygen), the gas-infused fluid may be removed from the fermenter via pathway 320, which optionally may include a filter 359, such as a carbon filter.

In some embodiments, the gas-infused fluid is treated with a second gas similar to the embodiments described with respect to FIG. 2. Accordingly, the system 300 may further comprise a second compressed gas source, which may be in communication with the gas infusion unit 308 substantially similar to the example configuration shown in FIG. 2. In such embodiments, the pressurized oxygen may be removed from the gas infusion chamber 308, the second gas may be introduced to the gas infusion chamber 308. Optionally, the fluid may be recirculated through the gas infusion chamber 308 in the presence of the second gas (e.g., at a pressure that is greater than atmospheric or ambient pressure) in order to quench any reactions occurring between the components of the fluid and the infused oxygen gas. The quenched gas-infused fluid may then be removed from the system 300 via product outlet 320.

Though example embodiments are described herein comprising a system and methods for infusing one or more gases (such as oxygen, nitrogen, or carbon dioxide) into an alcoholic beverage, the example embodiments are not limited to alcoholic beverages. As such, any fluid may be substituted for the alcoholic beverages of the present discussion. Such fluids may include, water, dairy products such as milk, cream, yogurts, colostrum, juices and ciders, sports or performance drinks, nutritional supplements, therapeutic fluids or other fluids.

Example implementations of the oxygen infusion process of the present invention can be used to reduce the concentration of certain congeners and other impurities in distilled spirits. In some cases, reducing the concentration of these congeners and other impurities reduces the perceptible taste of the spirit. This can be beneficial, for example, when producing spirits intended to have a subtle flavor profile (e.g., clear spirits such as vodka). In some cases, reducing the concentration of these congeners and other impurities can also alter the flavor profile of the spirit. This can be beneficial, for example, when modifying the flavor of a spirit in order to improve its aesthetic quality.

In some cases, implementations of the oxygen infusion process of the present invention can reduce the concentration of certain congeners and other impurities through esterification of fatty acids present in spirits. For example, after alcoholic fermentation, a spirit often includes varying concentrations of congeners such as hexadecanoic acids and octadecanonic acids. In many cases, hexadecanoic acids and octadecanonic acids are associated with a relatively harsh flavor profile. The oxygen infusion process of the present invention can convert all or some of the hexadecanoic acids and octadecanonic acids into the ethyl esters of each. In many cases, the resulting ethyl esters are associated with a relatively more pleasant flavor profile. As a result, the oxygen infusion process of the present invention can improve the flavor profile of the resulting distilled spirit.

Although two example congeners are described above, these are merely examples. In practice, implementations of the present invention also can be used to reduce the concentration of other congeners (e.g., isobutanol, amyl alcohols, propanol, and methanol) and/or convert harshly flavored congeners into more pleasantly flavored congeners, depending on the implementation. Further, the oxygen infusion process of the present invention also can be for other purposes as well, for example to reduce the presence of free radicals in the distilled spirit.

Accordingly, in some embodiments the present disclosure provides a system for producing a gas infused fluid. In some embodiments, the system comprises a compressed oxygen source, a gas infusion chamber for receiving the fluid, wherein the gas infusion chamber is in communication with the compressed oxygen cylinder, and a micromembrane in the gas infusion chamber, wherein the micromembrane has a pore channel diameter of about 0.05 μm to about 5.0 μm. In some embodiments, the fluid is a beverage. In some embodiments, the beverage comprises ethanol. In some embodiments, the beverage is a distilled spirit. In some embodiments, the distilled spirit is selected from the group consisting of: gin, rum, bourbon, cognac, tequila, whiskey, brandy, grappa, vodka, and a liqueur. In some embodiments, the beverage is beer. In some embodiments, the beverage is wine. In some embodiments, the beverage is a nutritional beverage. In some embodiments, the fluid is a therapeutic fluid. In some embodiments, the fluid comprises at least about 15 ppm of oxygen. In some embodiments, the fluid comprises at least about 25 ppm of oxygen. In some embodiments, the fluid comprises at least about 30 ppm of oxygen. In some embodiments, the fluid comprises at least about 50 ppm of oxygen. In some embodiments, the fluid comprises at least about 75 ppm of oxygen. In some embodiments, the fluid comprises at least about 100 ppm of oxygen. In some embodiments, the fluid comprises at least about 150 ppm of oxygen. In some embodiments, the fluid comprises at least about 200 ppm of oxygen. In some embodiments, the system further comprises a compressed nitrogen source. In some embodiments, the system further comprises a fluid supply pump in communication with the gas infusion chamber. In some embodiments, the system further comprises a holding chamber in communication with the gas infusion chamber. In some embodiments, the gas infusion chamber is housed within the holding chamber. In some embodiments, the system further comprises a supply pump in communication with the holding chamber and the gas infusion chamber. In some embodiments, the system further comprises a distiller in communication with the gas infusion chamber. In some embodiments, the system further comprises a chiller in thermal communication with a supply line, wherein the supply line is in fluid communication with the gas infusion chamber.

In another embodiment, the present disclosure provides a fluid comprising at least about 25 ppm of oxygen. In some embodiments, the fluid is a beverage. In some embodiments, the beverage comprises ethanol. In some embodiments, the beverage is a distilled spirit. In some embodiments, the distilled spirit is selected from the group consisting of: gin, rum, bourbon, cognac, tequila, whiskey, brandy, grappa, vodka, and a liqueur. In some embodiments, the beverage is beer. In some embodiments, the beverage is wine. In some embodiments, the beverage is a nutritional beverage. In some embodiments, the fluid is a therapeutic fluid. In some embodiments, the fluid comprises at least about 15 ppm of oxygen. In some embodiments, the fluid comprises at least about 30 ppm of oxygen. In some embodiments, the fluid comprises at least about 50 ppm of oxygen. In some embodiments, the fluid comprises at least about 75 ppm of oxygen. In some embodiments, the fluid comprises at least about 100 ppm of oxygen. In some embodiments, the fluid comprises at least about 150 ppm of oxygen. In some embodiments, the fluid comprises at least about 200 ppm of oxygen.

In another embodiment, the present disclosure provides a method of infusing oxygen in a distilled spirit, the method comprising containing a fluid within a gas infusion chamber, wherein the gas infusion chamber comprises a micromembrane having a pore channel diameter of about 0.05 μm to about 5.0 μm; pressurizing the gas infusion chamber with oxygen gas; contacting the fluid with the oxygen gas in the gas infusion chamber to provide an oxygenated fluid comprising at least about 25 ppm of oxygen; and removing the oxygenated fluid from the gas infusion chamber. In some embodiments, the step of pressurizing comprises pressurizing the gas infusion chamber to about 15 psi to about 100 psi. In some embodiments, the method further comprises circulating the fluid in the gas infusion chamber. In some embodiments, the step of circulating comprises passing the fluid from a holding tank through the gas infusion chamber a plurality of times. In some embodiments, the method further comprises releasing the oxygen gas from the gas infusion chamber and pressurizing the gas infusion chamber with a second gas. In some embodiments, the second gas is nitrogen or carbon dioxide. In some embodiments, the method further comprises releasing the second gas from the gas infusion chamber and pressurizing the gas infusion chamber with a third gas, wherein the third gas is different from the second gas. In some embodiments, the third gas is carbon dioxide or nitrogen. In some embodiments, the method further comprises heating the fluid. In some embodiments, the method further comprises cooling the fluid.

### Examples

### Example 1

In one example implementation of the present invention, a clear distilled spirit such as vodka was introduced to the gas infusion chamber 108 at room temperature and pure oxygen was delivered to the chamber at approximately 60 psi until oxygen saturation of the vodka reached a level of 160 ppm after 17 hours. Two taste tests were performed.

Taste Test #1: The first sampling was conducted after the vodka had been in the gas infusion chamber for 1 hour. The results from this taste comparison test demonstrated a noticeable improvement in the smoothness of the vodka as well as overall aroma (approx. a 20%-25% improvement). The aroma profile of the vodka prior to oxygen infusion was primarily sweet with vanilla undertones. In addition, the aroma contained a somewhat strong alcohol smell. The post oxygen infusion vodka aroma profile had noticeably less harsh alcohol smell and the flavor profile was noticeably smoother with a reduction in the strong bite or aftertaste.

Taste Test #2: The second sampling was conducted after the vodka had been in the oxygen infusion chamber for 20 hours. The results from this taste comparison test demonstrated a significant improvement in the flavor and aroma profile of the vodka (approx. a 90%-95% improvement in smoothness). The aroma profile had significantly less alcohol smell with reduced sweetness and vanilla. The flavor profile was significantly smoother with a nearly absent aftertaste or bite at the finish. The vodka profile was improved to the point where the product could be released as a limited edition vodka. A change to the processing time and amount of oxygen used in the Oxy-Aging process can achieve any desired combination of improvement in smoothness and altering the flavor profile.

### Example 2

In another example implementation of the present invention a dark spirit, such as a bourbon whiskey barrel aged for 14 months, was introduced to the gas infusion chamber 108 at room temperature and pure oxygen was delivered to the chamber at approximately 60 psi until oxygen saturation of the bourbon whiskey reached a level of 65 ppm after 20 hours. Two taste tests were performed. Two taste comparison tests were performed.

Taste Test #1: The first sampling was conducted after the bourbon had been in the oxygen infusion chamber for 1 hour. The results from this taste comparison test demonstrated a noticeable improvement in the flavor and aroma profile of the bourbon (approx. a 15%-20% improvement) post oxygen infusion. The aroma profile of the bourbon prior to oxygen infusion contained harsh alcohol and other volatile compounds typically associated with young bourbons. In addition, the aroma contained very strong earthy tones and over powering bourbon aromas. The post oxygen infusion aroma profile had noticeably less harsh alcohol and other volatile compounds smells. The flavor profile was noticeably smoother with a reduction in the acrid taste and unpleasant after taste of the bourbon.

Taste Test #2: The second sampling was conducted after the bourbon had been in the oxygen infusion vessel for 20 hours. The results from this taste comparison test demonstrated a significant improvement in the flavor and aroma profile of the bourbon (approx. a 70%-75% improvement) post oxygen infusion. The post oxygen infusion aroma profile had significantly less harsh alcohol and other volatile compounds present. In addition, the aroma of the bourbon was much smoother and more refined. The flavor profile was significantly smoother with an absence of any unpleasant or acrid taste. The bourbon profile was improved to the point where the product could be released for sale as mature bourbon.

### Example 3

Experience with oxygen infusion of distilled spirits using the system and processes of the present invention has resulted in the following observations regarding the change in the spirit's finish, flavor and aging profile.

Un-Aged Ethyl Alcohols: Un-aged spirits typically have a harsh, unpleasant taste. Its first and most obvious scent is fermented mash, such as corn, barley or other grain or starch, including acetaldehyde. Odors similar to furfural (a woody, sweet and almond-like scent) as well as coumarin (hay) abound. The product odor has some similarities to that of grain ethanol beer-well mixture (the fermented mash prior to first distillation). The taste is cloying and aldehydic, with strong notes of banana with the unpleasant burn of fusel oils. The flavor of several higher alcohols, propanols and butanols is evident. There is a lingering and unpleasant numbing aftertaste.

Oxygen Infusion Treatment: Post treatment of the unaged spirit using systems and processes of the present invention results in the product having a pleasant, yeasty odor of fresh-baked bread. Aldehydic flavors of grass and hay will be reduced, and the fruity odors will be fainter and more diverse and more delicate, likely due to a large increase in flavor esters. The taste will be pleasant and sweet and will compare favorably with better, aged corn whiskies. The harsh burn and aftertaste will be removed. We have developed a process that replicates what occurs in the traditional aging process that removes harsh chemicals from spirits.

The oxygen infusion process of the present invention results in the creation of new flavor compounds, mostly soft esters of the initial harsh congeners, a key benefit of the process. The process will treat even high molecular weight contaminants by in-situ chemical reaction, simultaneously reducing their concentration. As an example, the following objectionable fermentation by-products will be reduced in concentration:

2-methyl-1-propanol, 3-methyl-1-butanol (a fusel oil, and a cause of severe headaches)—some removed and some being esterified, 2-methyl-1-butanol (a fusel oil, and a cause of severe headaches)—some removed and some being esterified, ethyl acetate, 1-propanol, acetaldehyde, butanoic acid ethyl ester, capryic acid, isobutyl ester, and acetic acid heptyl ester.

During production of a distilled spirit, a fermentation process is commonly used to produce ethanol. Typically, this fermentation process also produces various congeners, such as other alcohols (e.g., fusel alcohols), acetone, acetaldehyde, esters, tannins, and aldehydes (e.g., propanol, furfural, glycols, and ethyl acetate). In many cases, congeners are responsible for the taste and aroma of resulting distilled spirit.

### Example 3

Commercial samples of gin, rum, bourbon, cognac, tequila mixto and tequila blanco were infused with oxygen by placing each spirit in a pressure vessel. Optionally, a nylon bag containing oak chips was also placed in the pressure vessel. After sealing the pressure vessel, the spirit was continuously circulated in the vessel by a circulation pump, and oxygen gas was introduced from a compressed oxygen source at a flow rate of 1.5 L/min to 3 L/min. Once the pressure vessel reached 60 psi (approximately 10-15 minutes), the circulation pump was turned off and activated once every 15 minutes. Additional oxygen was added to the pressure vessel as needed to maintain 60 psi in the pressure vessel. The resulting oxygen-infused spirits had an oxygen level of 160 ppm.

Each sample was analyzed by gas chromatography after 24 or 36 hours, as shown in Table 1.

TABLE 1
Gas chromatography analysis of commercial gin, rum, bourbon, cognac,
tequila mixto and tequila blanco.

| | | | | | | | | | DB |
|---|---|---|---|---|---|---|---|---|---|
| | | | | | | | | | Furfural |
| | Oxidation Notes | | | Fusel Oils | | | | | DB |
| Sample | MeCHO | EtOAc | MeOH | n-Pr* | iBu^{†} | 1-Bu^{‡} | IAA^{α} | AAA^{β} | Furfural |

RUM

| | | | | | | | | |
|---|---|---|---|---|---|---|---|---|
| Control | 17 | 27 | 8.2 | 52 | 41 | 3.1 | 109 | 22 |
| 24 h | 18 | 27 | 7.4 | 55 | 37 | 3.1 | 102 | 22 |
| % Change | 5.9% | 0.0% | −9.8% | 5.8% | −9.8% | 0.0% | −6.4% | 0.0% |

BOURBON

| | | | | | | | | |
|---|---|---|---|---|---|---|---|---|
| Control | 21 | 176 | 34 | 68 | 312 | 3.1 | 992 | 418 |
| 24 h | 39 | 176 | 38 | 65 | 322 | 1 | 1000 | 465 |
| % Change | 85.7% | 0.0% | 11.8% | −4.4% | 3.2% | −67.7% | 0.8% | 11.2% |
| 48 h | 37 | 170 | 39 | 69 | 326 | 1 | 1000 | 469 |
| % Change | 76.2% | −3.4% | 14.7% | 1.5% | 4.5% | −67.7% | 0.8% | 12.2% |
| 72 h | 37 | 167 | 40 | 60 | 316 | 1 | 1000 | 477 |
| % Change | 76.2% | −5.1% | 17.6% | −11.8% | 1.3% | −67.7% | 0.8% | 14.1% |
| 96 h | 35 | 162 | 37 | 62 | 315 | 1 | 1000 | 420 |
| % Change | 66.7% | −8.0% | 8.8% | −8.8% | 1.0% | −67.7% | 0.8% | 0.5% |
| 120 h | 38 | 168 | 40 | 67 | 323 | 1 | 1000 | 448 |
| % Change | 81.0% | −4.5% | 17.6% | −1.5% | 3.5% | −67.7% | 0.8% | 7.2% |
| 144 h | 38 | 166 | 38 | 65 | 314 | 2 | 1000 | 422 |
| % Change | 81.0% | −5.7% | 11.8% | −4.4% | 0.6% | −35.5% | 0.8% | 1.0% |

COGNAC

| | | | | | | | | |
|---|---|---|---|---|---|---|---|---|
| Control | 25 | 137 | 132 | 93 | 431 | 1.6 | 995 | 301 |
| 24 h | 48 | 123 | 115 | 115 | 475 | 1 | 1000 | 346 |
| % Change | 92.0% | −10.2% | −12.9% | 23.7% | 10.2% | −37.5% | 0.5% | 15.0% |
| 48 h | 42 | 121 | 113 | 115 | 469 | 1 | 1000 | 345 |
| % Change | 68.0% | −11.7% | −14.4% | 23.7% | 8.8% | −37.5% | 0.5% | 14.6% |
| 72 h | 1 | 1 | 123 | 120 | 468 | 1 | 1 | 1 |
| % Change | −96.0% | −99.6% | −6.8% | 29.0% | 8.6% | −37.5% | −99.9% | −99.7% |
| 96 h | 43 | 125 | 121 | 124 | 477 | 1 | 1000 | 334 |
| % Change | 72.0% | −8.8% | −8.3% | 33.3% | 10.7% | −37.5% | 0.5% | 11.0% |
| 120 h | 41 | 98 | 120 | 122 | 459 | 2 | 1000 | 340 |
| % Change | 64.0% | −28.5% | −9.1% | 31.2% | 6.5% | 25.0% | 0.5% | 13.0% |
| 144 h | 49 | 135 | 128 | 136 | 523 | 1 | 1000 | 360 |
| % Change | 96.0% | −1.5% | −3.0% | 46.2% | 21.3% | −37.5% | 0.5% | 19.6% |

TEQUILA MIXTO

| | | | | | | | | | |
|---|---|---|---|---|---|---|---|---|---|
| Control | 34 | 50 | 487 | 97 | 151 | 2.3 | 417 | 102 | 1.4 |
| 24 h | 24 | 50 | 442 | 82 | 134 | 4.4 | 439 | 97 | 1.84 |
| % Change | −29.4% | 0.0% | −9.2% | −15.5% | −11.3% | 91.3% | 5.3% | −4.9% | 31.4% |

TEQUILA BLANCO

| | | | | | | | | | |
|---|---|---|---|---|---|---|---|---|---|
| Control | 43 | 79 | 997 | 163 | 513 | 3.3 | 681 | 184 | 8.87 |
| 36 h | 37 | 66 | 896 | 163 | 435 | 2.3 | 569 | 165 | 10.49 |
| % Change | −14.0% | −16.5% | −10.1% | 0.0% | −15.2% | −30.3% | −16.4% | −10.3% | 18.3% |

WHITE WHISKEY

| | | | | | | | | |
|---|---|---|---|---|---|---|---|---|
| Control | 5.4 | 21 | 15 | 69 | 242 | 5.3 | 998 | 360 |
| 24 h Fr. Oak | 13 | 6.3 | 9.4 | 54 | 182 | 1.1 | 685 | 199 |
| % Change | 140.7% | −70.0% | −37.3% | −21.7% | −24.8% | −79.2% | −31.4% | −44.7% |
| 120 h Fr. Oak | 13 | 5 | 9 | 48 | 189 | 2 | 710 | 219 |
| % Change | 140.7% | −78.1% | −38.7% | −30.4% | −21.9% | −60.4% | −28.9% | −39.2% |

*n-Propanol;
^{†}Isobutanol;
^{‡}1-Butanol;
^{α}Isoamyl Alcohol;
^{β}Active Amyl Alcohol.

Analysis of white whiskey infused with oxygen in the presence of French Oak wood chips is shown in Table 2.

TABLE 2

| | | | | | | | | |
|---|---|---|---|---|---|---|---|---|
| trans- | cis- | | | | | | | |
| Lactones | Lactones | Vanillin | Eugenol | IE* | Guaiacol | 4-MG^{†} | Furfural | 5-MF^{‡} |

WHITE WHISKEY; French Oak

| | | | | | | | | | |
|---|---|---|---|---|---|---|---|---|---|
| 24 h | 299 | 348 | <50 | 17 | 2 | 44 | 20 | 0 | 1842 |
| 120 h | 19 | 301 | <50 | 12 | 4 | 10 | <5 | 243 | 4 |
| % Change | −93.6% | −13.5% | 0% | −29.4% | 100.0% | −77.3% | −75.0% | n/a | −99.8% |

*Isoeugenol;
^{†}4-Methylguaiacol;
^{‡}5-Methylfurfural.

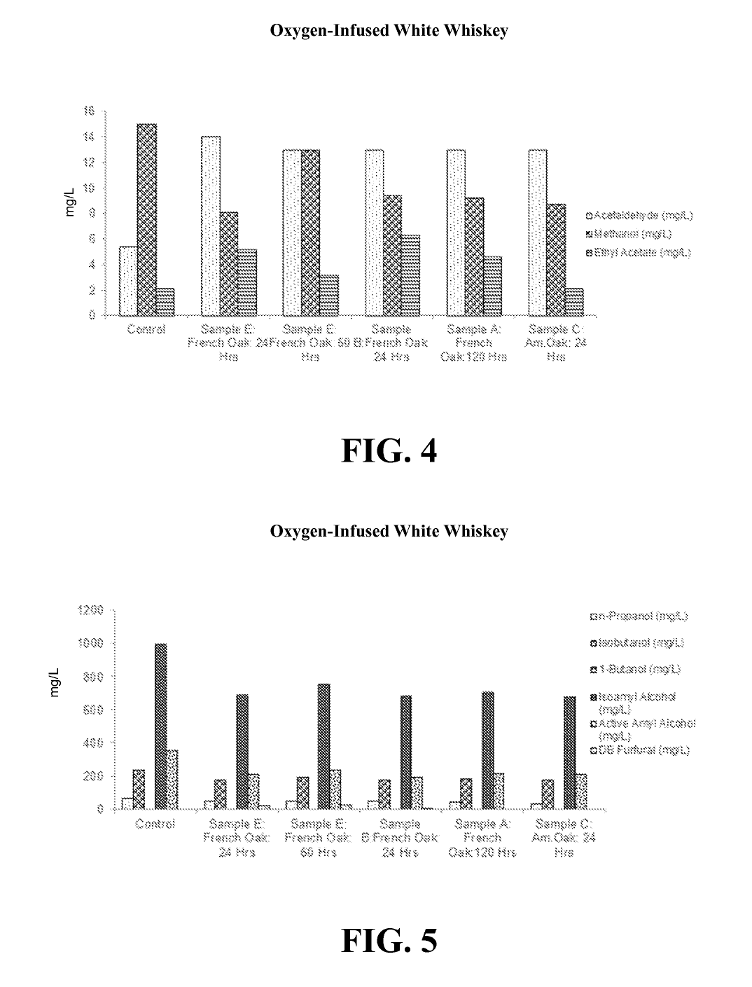

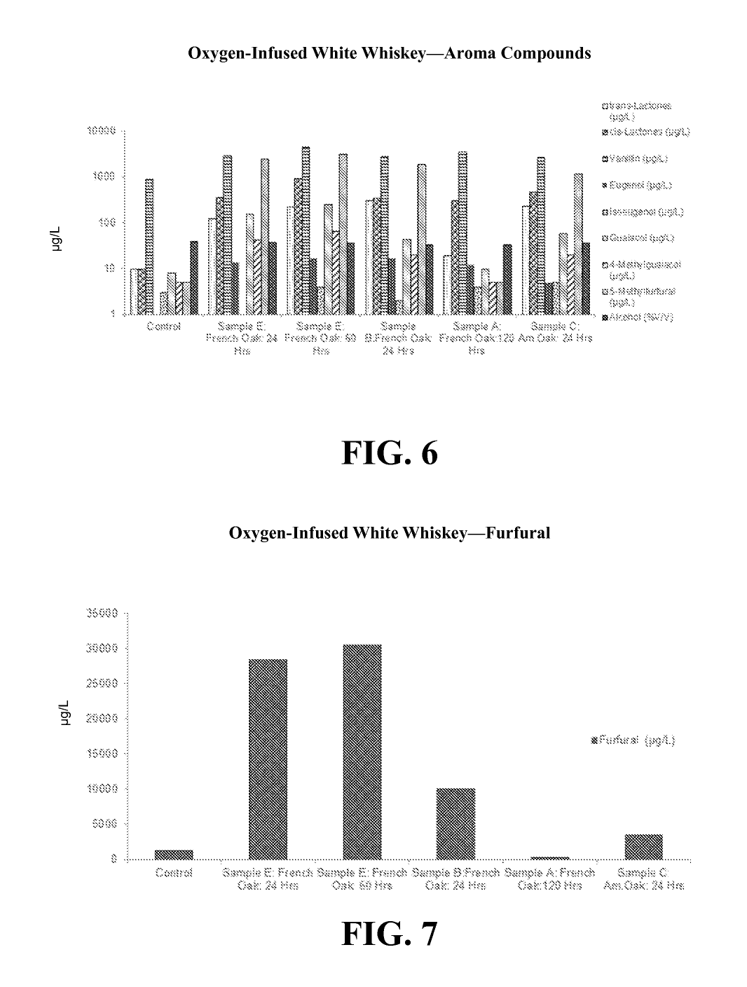

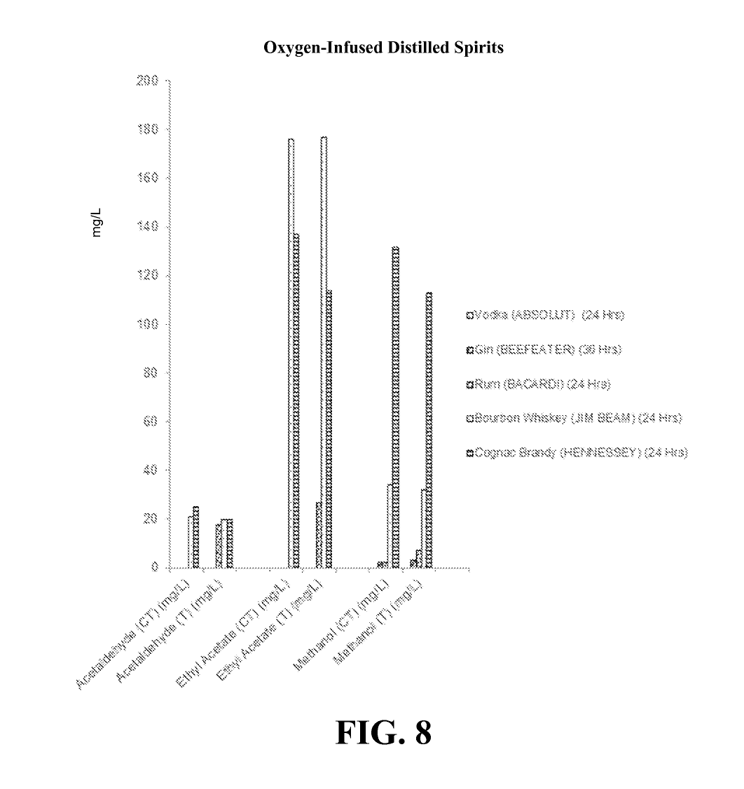

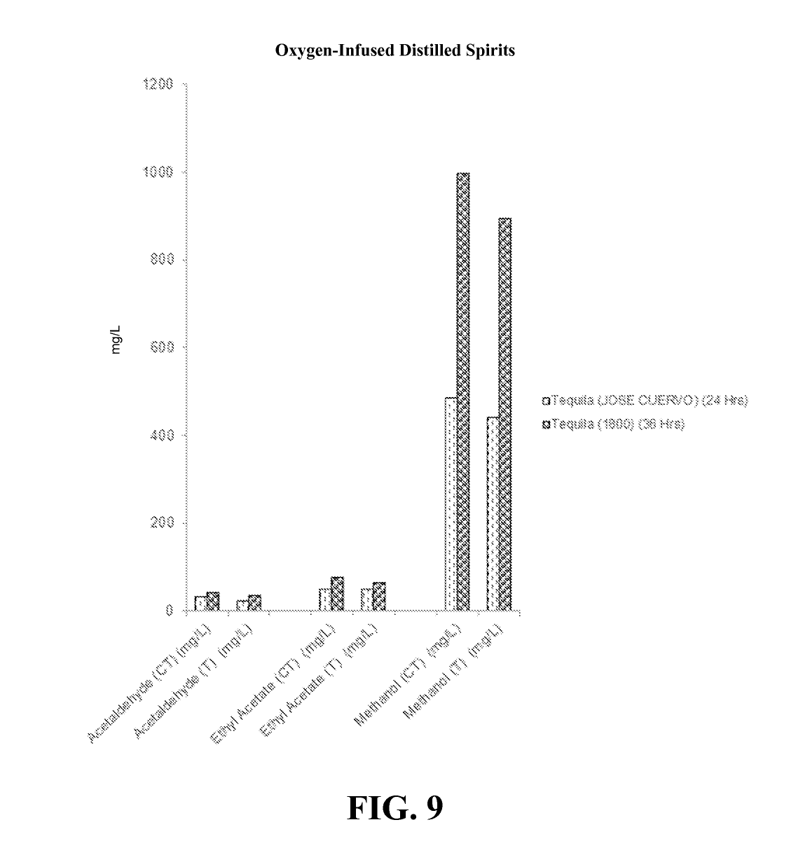

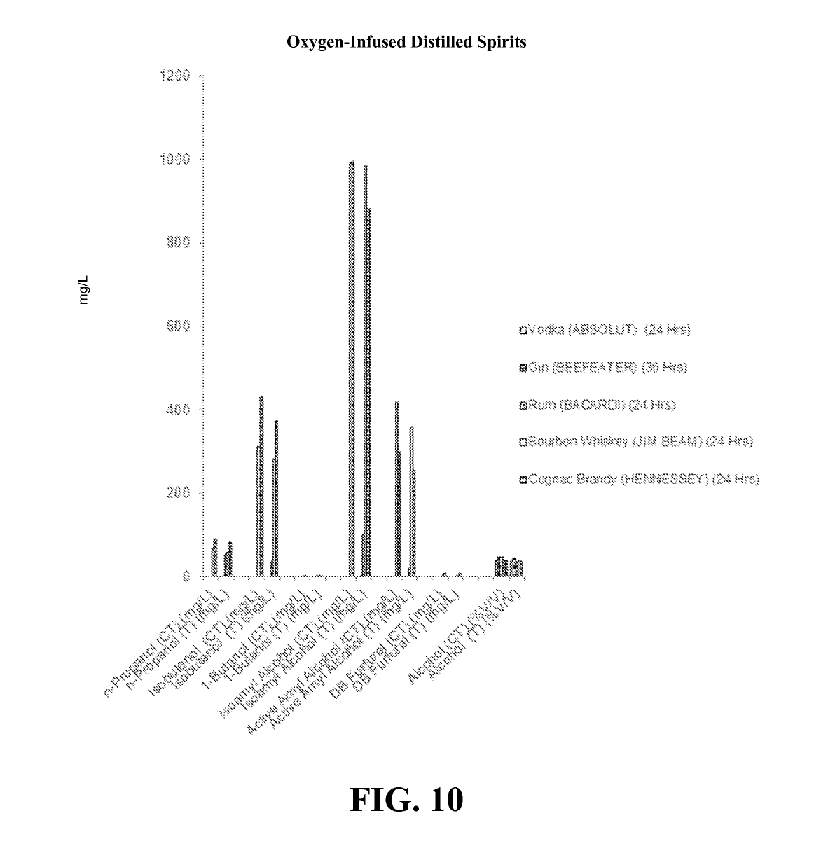

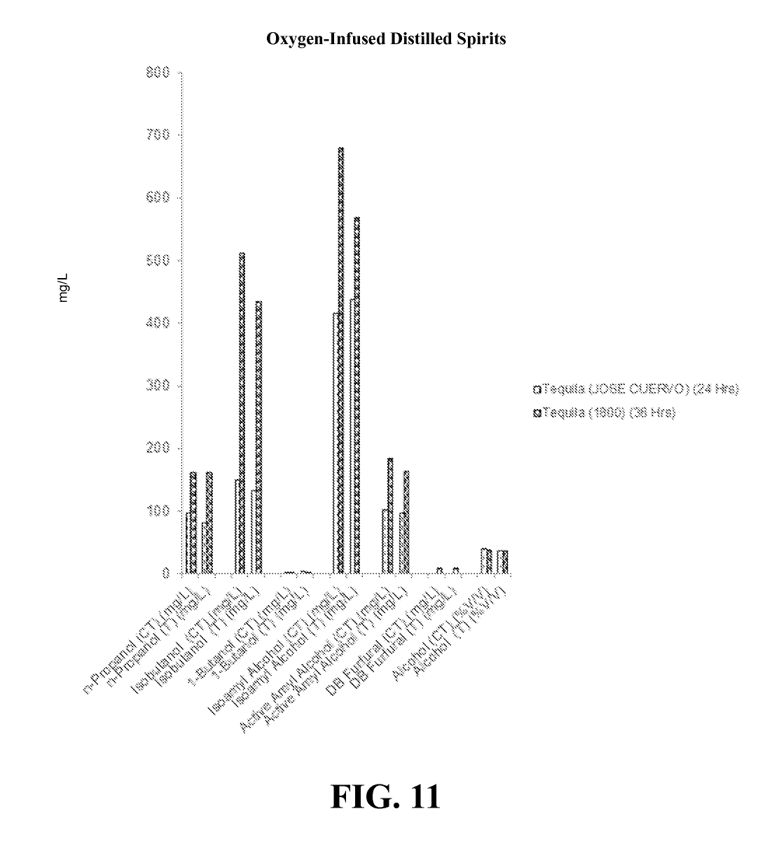

Similar data is shown in FIGS. 4 to 11 for commercially available distilled spirits infused with oxygen for 24 or 36 hours according to methods disclosed herein.

A number of embodiments of the invention have been described. Nevertheless, it will be understood that various modifications may be made without departing from the spirit and scope of the invention. For example, and with regard to dark spirits, the whiskey or rum can be aged for a time period in a barrel or cask (e.g., for approximately 1 to 36 months) and then processed in the oxygen infusion process described herein, and then returned to the barrel or cask for additional aging (e.g. approximately 1 to 60 months). In other embodiments of the present invention, aged distilled spirits, such as whiskeys aged in barrels or casks for 1 year or more including up to 30 years, can be treated with the oxygen infusion process described herein to further refine the finish and flavor profile. Accordingly, other embodiments are within the scope of the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method of infusing oxygen in a distilled spirit comprising;
(a) containing a fluid within a gas infusion chamber; wherein the gas infusion chamber comprises a micromembrane having a pore channel diameter of about 0.05 μm to about 5.0 μm;
(b) pressurizing the gas infusion chamber with oxygen gas;
(c) contacting the fluid with the oxygen gas in the gas infusion chamber to provide an oxygenated fluid comprising at least about 25 ppm of oxygen; and
(d) removing the oxygenated fluid from the gas infusion chamber.

42. The method of claim 41, wherein the step of pressurizing comprises pressurizing the gas infusion chamber to about 15 psi to about 100 psi.

43. The method of claim 41 further comprising circulating the fluid in the gas infusion chamber.

44. The method of claim 43, wherein the step of circulating comprises passing the fluid from a holding tank through the gas infusion chamber a plurality of times.

45. The method of claim 41 further comprising releasing the oxygen gas from the gas infusion chamber and pressurizing the gas infusion chamber with a second gas.

46. The method of claim 45, wherein the second gas is nitrogen or carbon dioxide.

47. The method of claim 45 further comprising releasing the second gas from the gas infusion chamber and pressurizing the gas infusion chamber with a third gas, wherein the third gas is different from the second gas.

48. The method of claim 47, wherein the third gas is carbon dioxide or nitrogen.

49. The method of claim 41 further comprising heating the fluid.

50. The method of claim 41 further comprising cooling the fluid.
